(19) United States
(12) Patent Application Publication
(10) Pub. No.: US 2005/0251231 A1
(43) Pub. Date: Nov. 10, 2005

(54) Treatment method and apparatus for basal cell carcinoma

(76) Inventor: Leonard H. Goldberg, Houston, TX (US)

Correspondence Address: LAW OFFICE OF DAVID MCEWING, P.O. BOX 231324, HOUSTON, TX, 77023, US

(21) Appl. No.: 10/838,490

(22) Filed: May 4, 2004

(51) Int. Cl. A01N 43/04; A61N 2/00; A61K 31/715; A61N 1/00

(52) U.S. Cl. 607/89; 606/2

(57) ABSTRACT

A method and device for treatment of basal cell carcinoma utilizing controllable and substantially monochromatic electromagnetic radiation, such as emitted by a pulsed ray laser. The wavelength may range from 585 nm to 1560 nm.

### BACKGROUND OF INVENTION

1. Field of Use

The invention subject of this invention pertains to a laser removal of the basal cell carcinoma. The present invention is a method and device for selective photothermolysis of selected surgical targets. Specifically, the present invention can be used to remove basal cell carcinoma tumors with minimal damage to the surrounding healthy tissue.

2. Background of the Invention

Skin cancer is the most common type of cancer with more than 1 million cases diagnosed each year in the United States. About 97% of all skin cancer is nonmelanoma skin cancer. Basal cell carcinoma (BCC) is the most common type of skin cancer and accounts for 80% of all nonmelanoma skin cancer.

Treatment modalities of basal cell carcinoma (BCC) include electrodessication and cureftage, cryotherapy, excision, and Mohs micrographic surgery. New treatments that have been studied include topical imiquimod, topical 5-fluorouracil, and the carbon dioxide laser. The pulsed-dye laser selectively destroys blood vessels using hemoglobin as a chromophore and has been used for the treatment of vascular lesions such as port-wine stain, hemangioma, and telangiectasia.

Selective photothermolysis is a known surgical method for destroying certain diseased or unsightly tissue, on or near the skin, with minimal damage to the surrounding healthy tissue. This method has been used for treatment of birthmarks, port-wine stains, spider veins, and varicose veins, all of which tend to be much redder than the surrounding tissue because of their higher concentration of oxyhemoglobin-containing red blood cells. The method consists of irradiating the target and the surrounding tissue with pulsed electromagnetic radiation, usually visible radiation that is preferentially absorbed by the target. The tissue to be destroyed must be characterized by significantly greater optical absorption at some wavelength of electromagnetic radiation than the surrounding tissue. The energy and duration of the pulses is such that the target is heated to between about 70.degree. C. and about 80.degree. C., at which temperature the proteins of the target coagulate. Because the target absorbs the incident radiation much more strongly than the surrounding tissue, the surrounding tissue is heated negligibly. Past work and studies have indicated radiation having wavelengths of 577 nm and 585 nm to be useful in the treatment of birthmarks or portwine stain.

The preferable radiation source is a laser, for example a flashlamp-pulsed dye laser. A laser source has the advantage of being inherently monochromatic. This has the advantage of minimizing the unintended or undesired induction of heat within healthy tissue. The prior art has recognized that one constraint on the pulse laser radiation treatment has been that the pulse duration may not be of such length as to cause the surrounding tissue to be heated to the point that it, too, begins to coagulate. As the target is heated, heat begins to diffuse from the target to the cooler surrounding tissue. To keep the surrounding tissue from being heated to the point of damage, the pulse length must be kept on the order of the target's thermal relaxation time. For relatively small targets, such as birthmarks, port-wine stains, and spider veins, typical pulse lengths are on the order of hundreds of microseconds. For varicose veins, pulse lengths on the order of milliseconds should be used. The need to cool the epidermal area prior to pulsed light treatment is the subject of U.S. Pat. No. 5,814,040 issued to J. Stuart Nelson et al and U.S. Pat. No. 6,514,244 issued to Karl Pope et al. The goal of cooling is to establish a steep temperature gradient between the skin surface and the target tissue.

Difficulties is achieving the desired cooling have been encountered. Although spraying of a cryogen is known (dynamic cooling) problems with “flame like” light flashes have been encountered. Modifications to the dynamic cooling methodology and apparatus have variously been disclosed.

Further, U.S. Pat. No. 6,214,034, issued to Zion Azar, discloses a method and apparatus of selective photothermolysis of a target tissue within a surrounding tissue. The target and the surrounding tissue are heated to a predetermined temperature of about 60° C. by a pulsed heat source such as a flash lamp thereby creating a temperature gradient in the air included in a cavity formed between the housing of the apparatus and the surrounding tissue. When the tissue surface reaches the predetermined temperature the target tissue is heated to the point of coagulation, preferably by narrow band electromagnetic radiation. The temperature difference between the coagulation target and the surrounding tissue is sufficiently mild that heat diffusing out of the target does not damage the surrounding tissue, even in the case of a relatively large target such as varicose veins. Azar teaches synchronization of the monochromatic pulses with the broad-band electromagnetic radiation, by means well-known in the art, to ensure that the surrounding tissue has been heated sufficiently before the monochromatic pulse is turned on to heat the target further, and to ensure that the target is heated further before the surrounding tissue has a chance to cool down.

### SUMMARY OF INVENTION

The method subject of this invention pertains to the use of 595 nm pulsed-dye laser for superficial basal cell carcinoma. The invention has demonstrated effective treatment response, i.e., a clinical and histologic clearance (100%) for superficial BCC residual tumor. This treatment response was achieved within the operational parameters of 595 nm wavelength, at a fluence of 15 J/cm² without epidermal cooling mode and pulse duration of 1.5 ms to 3 ms (3×10⁻³ seconds). These results were obtained with a commercial V-beam vascular lesion laser manufactured by Candela Corporation of Wayland, Mass.

In addition to the use of a pulse beam laser, the invention includes an alternate means for generating the substantially monochromatic radiation used to heat a target. This alternate means is to pass light from the high intensity lamp through a suitable wavelength selection device, such as a narrow band filter or a monochromator.

The mechanism for operability is the redness of the BCC tumor caused by increased blood supply to the area of rapidly dividing cells. The darker color causes an increased absorption of radiation with a resulting increase in cell temperature. The radiation energy preferentially absorption of by the hemoglobin, which is the major chromophore in the blood in the ectatic capillaries in the upper dermis, is converted to heat, causing thermal damage and thrombosis in the targeted tissue.

### SUMMARY OF DRAWINGS

The accompanying drawings, which are incorporated in and constitute a part of the specification, illustrate preferred embodiments of the invention. These drawings, together with the general description of the invention given above and the detailed description of the preferred embodiments given below, serve to explain the principles of the invention.

FIG. 1 illustrates the prior apparatus used in the laser treatment of skin tissue, including basal cells, with a cryogen supply connected to the hand application component.

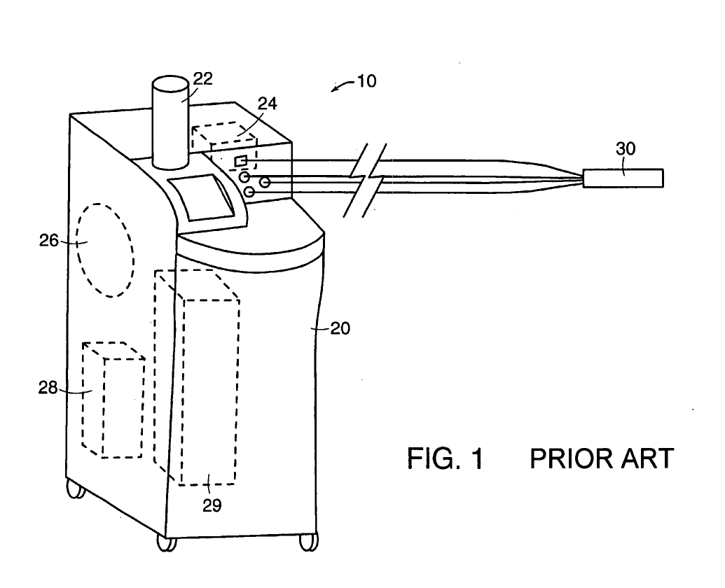

FIG. 2 illustrates the prior art hand application component incorporating the light applicator and cryogen applicator.

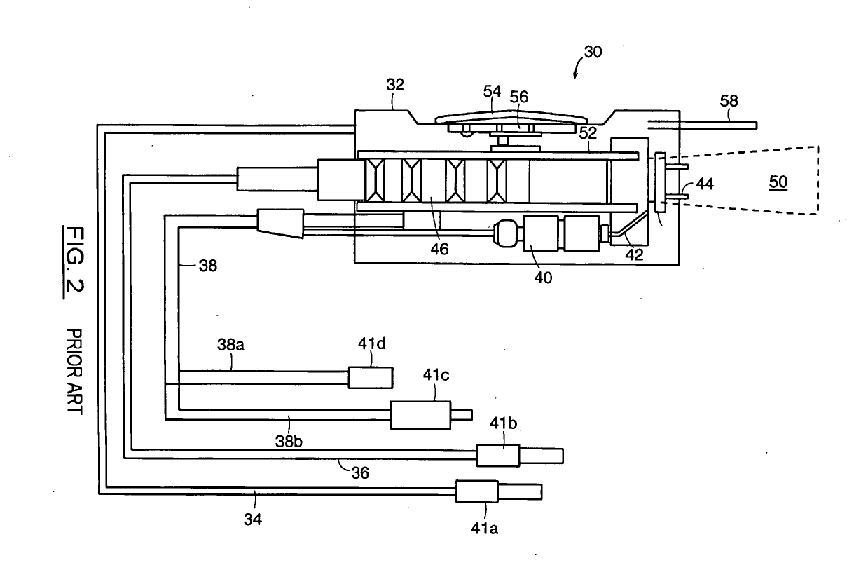

FIG. 3 illustrates the steps of the prior art requiring application of cryogenic fluid prior to irradiation of the tissue.

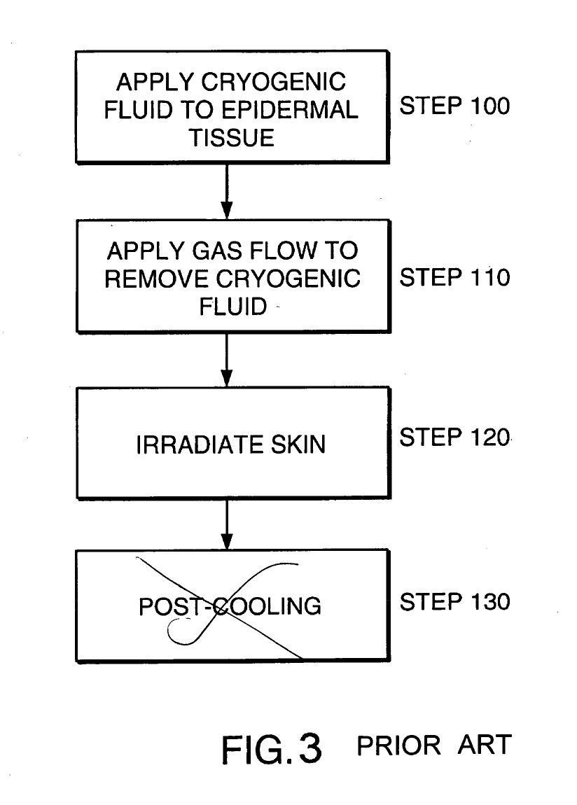

FIG. 4 represents the steps of the preferred embodiment of the present invention.

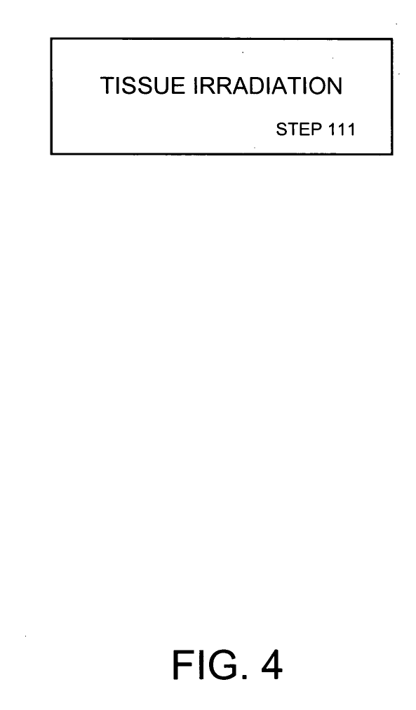

### DETAILED DESCRIPTION OF THE INVENTION

The above general description and the following detailed description are merely illustrative of the subject invention, and additional modes, advantages and particulars of this invention will be readily suggested to those skilled in the art without departing from the spirit and scope of the invention.

The device of the present invention accomplishes this end by heating the hemoglobin within the blood vessels, causing the blood vessels to coagulate with venosity necrosis of the tumor. The scope of the present invention includes all effective wavelength of electromagnetic radiation, and effective spectral bands for this purpose include microwave radiation; but the preferred spectral band, for the for heating the surrounding tissue and for heating the target itself, is 595 to 1560 nm. The preferred device for generation this light is a pulsed dye laser emitting 585 to 595 nm of light but laser light from 585 to 1560 nm may be suitable. The device includes a mechanism for pulsing the light from the light source. This mechanism may include circuitry for controlling the current supplied to the light source by either power or a shutter.

Materials and methods: Superficial BCCs were exposed to a 595 nm pulsed-dye laser (V-Beam laser, Candela Corp., Wayland, Mass.), using 4 sets of laser parameters varying in fluence (J/cm²) and epidermal cooling mode (DCD on/off): A) 3 J/cm², DCD on, B) 7 J/cm², DCD on, C) 15 J/cm², DCD on, and D) 15 J/cm², DCD off. One pass of laser with 10% overlapping pulses, pulse duration of 1.5 ms to 3 msec, spot size 7 mm, and 4 mm treatment margins were used in all groups. At 3-8 weeks after laser treatment, the treated superficial BCCs were clinically evaluated, excised, and examined histologically (H&E) using serial section. Clinical response was evaluated as: no change, flattening, less erythema, or absence of apparent residual tumor. Therapeutic response was determined by histological findings and graded as: failure (tumor seen) or success (no tumor seen).

Results: There were 25 superficial BCCs; 4 in group A, 5 in group B, 6 in group C, and 10 in group D. The location of the tumors included the shoulder (9), back (8), chest (4), arms (2), and legs (2). The average follow-up period was 5.3 (3-8) weeks.
Immediate clinical findings: Group A: no change (100%), group B: slightly erythema (20%), and purpura (80%), group C: purpura (100%), and group D: purpura, swelling, and epidermal grayish whitening (100%).
Clinical response: Group A: no change (100%), group B: no change (60%), and slight flattening and less erythema (40%), group C: marked flattening and less erythema (33.3%), and slight flattening and less erythema (66.7%), and group D: absence of apparent residual tumor (100%).
Therapeutic/histological response: Histologically, no tumor was seen after treatment in 0% of groups A and B, 20% of group C, and 100% of group D.

Conclusion: The 595 run pulsed-dye laser, at a fluence of 15 J/cm2 without epidermal cooling mode, provides a significant therapeutic response with clinical and histologic clearance for superficial BCC.

It will be appreciated that the most success was achieved without prior epidermal cooling with the highest intensity (J/cm² ) electromagnetic radiation. The invention has demonstrated also that treatment of BCC is enhanced when the tissue surface is at normal ambient temperature, approximately 30° C., prior to the application of the electromagnetic radiation of 595 nm. This is contrary to the teaching of the prior art wherein cryogen is used to cool the ambient skin temperature to approximately minus 10° C. The present invention has demonstrated that maintaining the tissue and target tissue at normal temperature is beneficial. This may be the result of there being a smaller temperature gradient between the surrounding tissue and the target tissue at the start of the treatment. Accordingly, the treatment will not create the dramatic temperature gradient between the target tissue, heated by radiation energy, and the surrounding tissue that does not experience significant absorption of radiation.

The absence of cryogen will also avoid the undesired light flash.

This specification is to be construed as illustrative only and is for the teaching those skilled in the art the manner of carrying out the invention. It is to be understood that the forms of the invention herein shown and describe are to be taken as the presently preferred embodiments. As already stated, changes, variations or refinements may be made to the method without departing from the scope of this invention. For example, equivalent elements may be substituted for those illustrated and described herein and certain features of the invention may be utilized independently of the use of other features, all as would be apparent to one skilled in the art after having the benefit of this description of the invention.

1. A method for treatment of basal cell carcinoma comprising the steps of:
a) Subjecting dermal tissue containing a basal cell carcinoma to electromagnetic radiation comprised substantially of a single wavelength within a range of between 585 nm to 1560 nm and a fluence of at least 7 J/cm²; and
b) irradiating the tissue for a period of 1.5 to 3 msec.

2. The invention of claim 1 further comprising using electromagnetic radiation having a substantially single wavelength of approximately 595 nm.

3. The invention of claim 1 further comprising electromagnetic radiation having a fluence of 15 J/cm².

4. The invention of claim 1 wherein the dermal tissue is at a normally maintained body temperature prior to irradiation.

5. A method of treatment of basal cell carcinoma comprising the steps of
a) establishing a skin surface temperature of proximate to a target dermal tissue of approximately 30° C.;
b) irradiating the dermal tissue with electromagnetic radiation comprised substantially of a single wavelength of approximately 595 nm at a fluence of 15 J/cm² for approximately 3 msec.

6. The method of claim 5 further comprising using a pulsed-dye laser,

7. An apparatus for treatment of basal cell carcinoma comprising
a) an electromagnetic radiation source producing electromagnetic radiation of substantially a single wavelength variably of between 585 nm and 1560 nm;
b) means to emit the electromagnetic radiation to a directed location of a skin surface and at a fluence of between 6 and 16 J/cm²; and
c) means to control the emission of the electromagnetic radiation to the skin surface between 1.4 and 3.1 msec.

8. The apparatus of claim 7 further wherein the wavelength is approximately 595 nm.
